(19) United States
(12) Patent Application Publication
(10) Pub. No.: US 2006/0229542 A1
(43) Pub. Date: Oct. 12, 2006

(54) Orthosis

(76) Inventor: Norman D. Sinreich, Westlake, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/399,545

(22) Filed: Apr. 6, 2006

Related U.S. Application Data
(60) Provisional application No. 60/668,672, filed on Apr. 6, 2005.

(51) Int. Cl. A61F 5/00

(52) U.S. Cl. 602/27

(57) ABSTRACT

An orthosis connectable with a patient includes a first portion connectable with a leg of the patient. A second portion is connectable with a foot of the patient. The second portion is movable relative to the first portion about a pivot axis spaced from a plantar/dorsal pivot axis of the patient. A control device controls relative movement between the first and second portions about the pivot axis. The control device is connected with an anterior portion of the first portion and an upper portion of the second portion.

### RELATED APPLICATION

The present application claims priority from U.S. Provisional Application No. 60/668,672, filed Apr. 6, 2005 herein incorporated by reference in its entirety.

### FIELD OF THE INVENTION

The present invention relates to an orthosis, and more specifically, to an ankle-foot orthosis.

### BACKGROUND OF THE INVENTION

An ankle-foot orthosis generally includes a leg portion connectable with a patient's leg and a foot portion connectable with a patient's foot. The leg portion and the foot portion are interconnected by a hinge. An axis of the hinge is aligned with the plantar/dorsal axis of ankle of the patient. The trim lines for the leg and foot portions must be carefully fitted to the patient's leg. The leg and foot portions may need to be cut to adjust the fit and function of the orthosis. After cutting the leg and foot portions, one may determine that too much material was removed. If too much material is removed, new leg and foot portions must be made.

### SUMMARY OF THE INVENTION

An orthosis of the present invention includes a first portion connectable with a leg of a patient. A second portion is connectable with a foot of the patient. The second portion is movable relative to the first portion about a pivot axis spaced from a plantar/dorsal pivot axis of the patient. A control device controls relative movement between the first and second portions about the pivot axis. The control device is connected with an anterior portion of the first portion and an upper portion of the second portion.

### BRIEF DESCRIPTION OF THE DRAWINGS

The foregoing and other features of the present invention will become apparent to one skilled in the art to which the present invention relates upon consideration of the following description of the invention with reference to the accompanying drawings, in which:

FIG. 1 is a schematic pictorial view of an exemplary orthosis including a first embodiment of a control device;

FIG. 2 is a schematic pictorial view of the orthosis of FIG. 1 showing the orthosis connected with a patient's leg;

FIG. 3 is a schematic pictorial view of a brace member of the orthosis of FIG. 1;

FIG. 4 is a schematic plan view of a second embodiment of a control device for use with the brace member of FIG. 3;

FIG. 5 is a schematic side view of the control device of FIG. 4;

FIG. 6 is a schematic pictorial view of a third embodiment of a control device for use with the brace member of FIG. 3 showing the control device in a first position; and

FIG. 7 is a schematic pictorial view, similar to FIG. 6, showing the control device of FIG. 6 in a second position;

### DESCRIPTION OF THE INVENTION

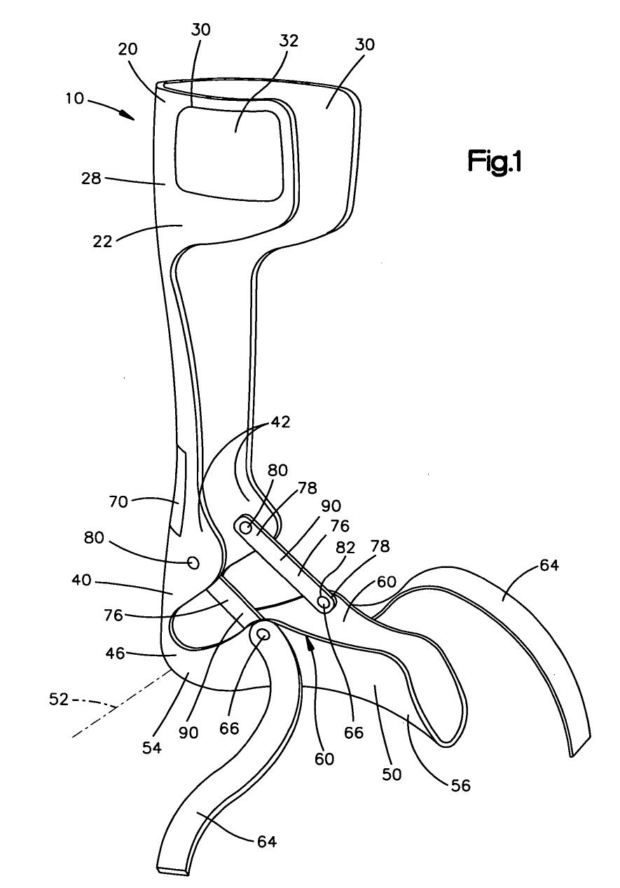

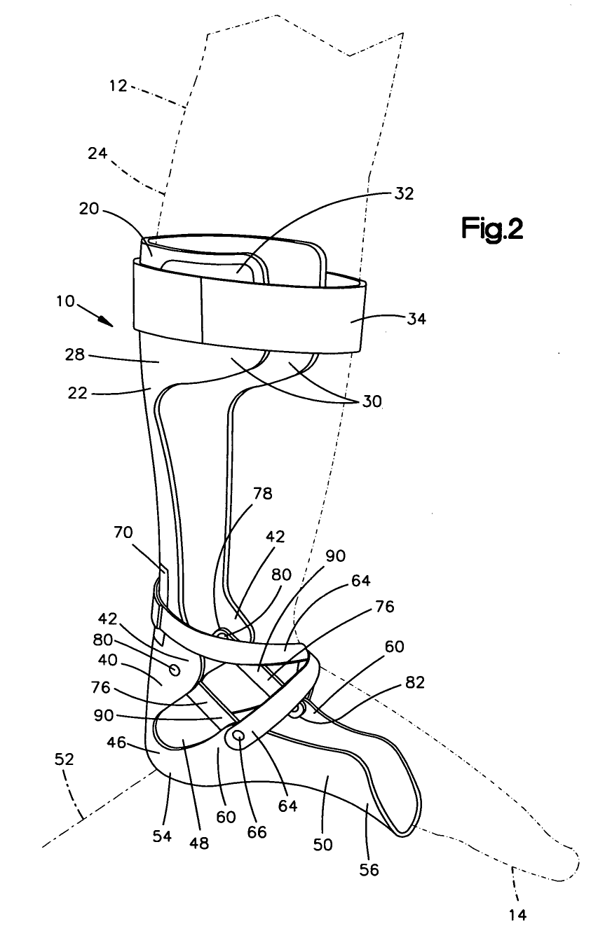

An orthosis or brace 10 constructed in accordance with an exemplary embodiment of the present invention is schematically illustrated in FIGS. 1 and 2. The orthosis 10 may be connected to a leg 12 of a patient to control plantar flexion and/or dorsiflexion movement of a foot 14 of the patient. The orthosis 10 may resist, stop, prevent, or permit free plantar flexion. The orthosis 10 may resist, stop, prevent, or permit free dorsiflexion. The orthosis 10 may be weight bearing and help control ground reaction forces. The orthosis 10 may also be worn inside a shoe.

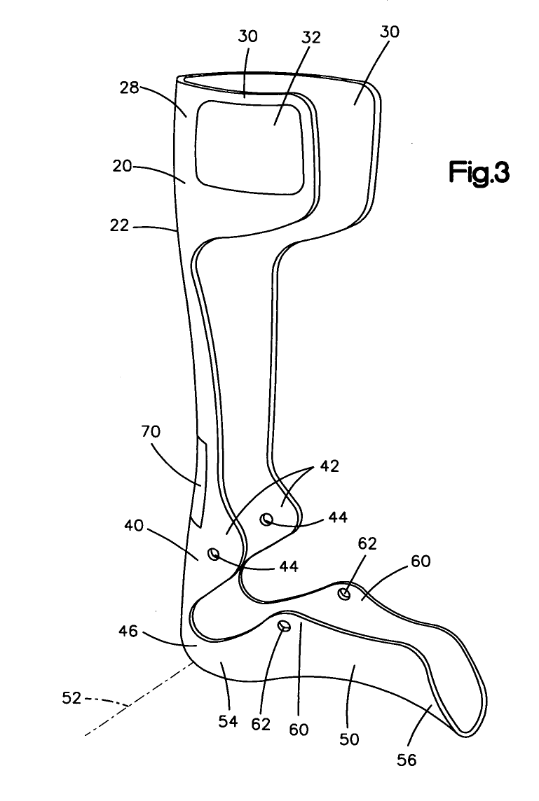

The orthosis 10 may include a one-piece brace member 20 (FIGS. 1-3) having a first or leg portion 22 connectable with the leg 12 of the patient. The leg portion 22 may generally extend along a posterior portion 24 of the leg 12. An upper end 28 of the leg portion 22 may include support portions 30. The support portions 30 may extend on opposite sides of the leg 12 to connect the leg portion 22 to the leg.

The support portions 30 may include first parts 32 of hook and loop fasteners. A strap 34 (FIG. 2) may include second parts of the hook and loop fasteners. The strap 34 may extend around the leg 12 and be connected to the first parts 32 to connect the leg portion 22 to the leg 12. It is contemplated that the leg portion 22 may be connected to the leg 12 of the patient in any desired manner. It is also contemplated that the leg portion 22 may include padding for engaging the leg 12.

The leg portion 22 may include a lower end 40 (FIGS. 1-3) opposite the upper end 28. The lower end 40 may generally extend along the posterior portion 24 of the leg 12 of the patient. The lower end 40 may include anterior portions or flanges 42 with openings 44. The flanges 42 may extend on opposite sides of the leg 12. The flanges 42 may be substantially spaced from the axis of plantar/dorsal motion of the patient.

A resilient connecting portion 46 may extend from the lower end 40 of the leg portion 22 around a heel 48 of the patient. The connecting portion 46 may interconnect the leg portion 22 and a foot portion 50. The connecting portion 46 may be substantially spaced from the axis of plantar/dorsal motion of the patient. The connecting portion 46 may permit relative movement between the leg portion 22 and the foot portion 50 about a pivot axis 52. The pivot axis 52 may extend generally parallel to and be spaced from the axis of plantar/dorsal motion of the patient. The pivot axis 52 may be posterior and inferior to the axis of plantar/dorsal motion. Accordingly, the connecting portion 46 may act as a hinge between the leg portion 22 and the foot portion 50. Although the connecting portion 46 is shown extending around the heel 48, any desired connecting portion that permits the foot portion 50 to move relative to the leg portion about a pivot axis spaced from the axis of plantar/dorsal motion may be used.

The foot portion 50 may be connected to the foot 14 and extend generally under the foot. A first posterior end 54 of the foot portion 50 may be connected to the connecting portion 46. The foot portion 50 may include a second anterior end 56 opposite the first end 54. The foot portion 50 may include upper portions or flanges 60 with openings 62. The flanges 60 may extend on opposite sides of the foot 14. The flanges 60 may be substantially spaced from the axis of plantar/dorsal motion of the patient. The leg portion 14, connecting portion 46 and foot portion 50 may be formed as one-piece in any desired manner.

Connecting straps or elements 64 (FIGS. 1 and 2) may be pivotally connected to the flanges 60 by fasteners 66 extending through the openings 62. The lower end 40 of the leg portion 22 may include first parts 70 of hook and loop fasteners. The straps 64 may include second parts of the hook and loop fasteners. The straps 64 may cross over the foot 14 and extend around the leg 12 of the patient and be connected to the first parts 70 to connect the foot portion 50 to the foot 14. A shoe may be worn over the foot portion 50 to help connect the foot portion to the foot 14. It is contemplated that the foot portion 50 may be connected to the foot 14 in any desired manner. It is also contemplated that the foot portion 50 may include padding for engaging the foot 14.

Control devices 76 (FIGS. 1-2) for controlling relative movement between the leg portion 22 and the foot portion 50 may extend on opposite sides of the leg 12. The control devices 76 may resist, stop, prevent, or permit free movement of the foot portion 50 about the pivot axis 52 relative to the leg portion 22 in a first direction, such as counterclockwise, as viewed in FIGS. 1 and 2, or dorsiflexion. The control devices 76 may resist, stop, prevent, or permit free movement of the foot portion 50 about the pivot axis 52 relative to the leg portion 22 in a second direction, such as clockwise, as viewed in FIGS. 1 and 2, or plantar flexion. It is contemplated that any desired control devices 76 may be used to control relative movement between the leg portion 22 and the foot portion 50.

The control devices 76 may have first ends 78 pivotally connected to the anterior portions or flanges 42 of the leg portion 22 by the fasteners 80. The first ends 78 may be connected to the anterior portions 42 at locations that are proximal to the plantar/dorsal axis of the patient. The control devices 76 may have second ends 82 pivotally connected to the upper portions or flanges 60 of the foot portion 50 by the fasteners 66. The second ends 82 may be connected to the upper portions 60 at locations that are distal to the plantar/dorsal axis of the patient.

The first and second ends 78 and 82 of the control devices 76 may move relative to each other upon relative movement between the leg portion 22 and the foot portion 50. The first and second ends 78 and 82 of the control devices 76 may pivot relative to the anterior portions 42 of the leg portion 22 and the upper portions 60 of the foot portion 50 upon relative movement between the leg portion and the foot portion. the control devices 76 may be connected to inner surfaces of the brace member 20, as shown in FIGS. 1-2. The control devices 76 may be connected to outer surfaces of the brace member 20. It is also contemplated that the brace member 20 may include recesses for receiving the first and second ends 78 and 82 of the control devices 76.

A first embodiment of an exemplary control device 76 is illustrated in FIGS. 1-2. The control device 76 may be a flexible member 90. The flexible member 90 may be any desired flexible member, such as a strap of fabric. The member 90 may permit free movement of the foot portion 50 relative to the leg portion 22 from a predetermined position in a first direction, such as dorsiflexion or counterclockwise, as viewed in FIGS. 1 and 2. The member 90 may prevent movement of the foot portion 50 relative to the leg portion 22 from the predetermined position in a second direction opposite the first direction, such as plantar flexion or clockwise, as viewed in FIGS. 1 and 2.

The predetermined position may be any desired position. The predetermined position may be partially defined by the member 90. The member 90 may be connected to the leg portion 22 and the foot portion 50 so that the member is in a fully extended position to define the predetermined position. The member 90 may be in the fully extended position when the foot portion 50 is in a dorsiflexed position, a neutral position, or a plantar flexed position.

The member 90 may be connected to the leg portion 22 and the foot portion 50 so that the flexible member is in a slacked position when the foot portion is in the predetermined position relative to the leg portion 22. The predetermined position may be the neutral position. The member 90 may permit free movement of the foot portion 50 relative to the leg portion 22 from the predetermined position in the first direction, such as dorsiflexion or counterclockwise, as viewed in FIGS. 1 and 2. The member 90 may permit free movement of the foot portion 50 relative to the leg portion 22 from the predetermined position in the second direction, such as plantar flexion or clockwise, as viewed in FIGS. 1 and 2 until the member is fully extended. The member 90 may prevent or stop further movement of the foot portion 50 relative to the leg portion 22 in the second direction when the member is fully extended. Accordingly, the member 90 may be a stop to further movement of the foot portion 50 in the second direction.

The member 90 of the control device 76 may be a flexible member, such as an elastic member. The member 90 may be any desired member that applies any desired force to urge the foot portion 50 to move in the first direction relative to the leg portion 22 upon stretching of the elastic member. The member 90 may permit free movement of the foot portion 50 relative to the leg portion 22 from the predetermined position in the first direction, such as dorsiflexion or counterclockwise, as viewed in FIGS. 1 and 2. The member 90 may resist or restrict movement of the foot portion 50 relative to the leg portion 22 from the predetermined position in the second direction, such as plantar flexion or clockwise, as viewed in FIGS. 1 and 2 until the member is fully extended. The member 90 may stretch and apply a force between the leg portion 22 and the foot portion 50 during movement of the foot portion in the second direction to resist or restrict movement in the second direction. The member 90 may urge the foot portion 50 to move in the first direction when the member stretches. The member 90 may prevent or stop further movement of the foot portion 50 relative to the leg portion 22 in the second direction when the member is fully extended.

If the member 90 is an elastic member, the member 90 may be connected to the leg portion 22 and the foot portion 50 so that the member is in a slacked position when the foot portion is in the predetermined position relative to the leg portion 22. The member 90 may permit free movement of the foot portion 50 relative to the leg portion 22 from the predetermined position in the first direction, such as dorsiflexion or counterclockwise, as viewed in FIGS. 1 and 2. The member 90 may permit free movement of the foot portion 50 relative to the leg portion 22 from the predetermined position in the second direction, such as plantar flexion or clockwise, as viewed in FIGS. 1 and 2 until the member begins to stretch and apply a force between the leg portion 22 and the foot portion 50 to resist or restrict movement. The member 90 may prevent or stop further movement of the foot portion 50 relative to the leg portion 22 in the second direction when the member is fully extended. Accordingly, the member 90 may be a stop to further movement of the foot portion 50 in the second direction.

It is contemplated that the control device 76 may include any desired number of flexible members 90. It is also contemplated that the control device 76 may include a first flexible member, such as a strap of fabric, to act as a stop member and a second elastic member to resist or restrict movement of the foot portion 50 relative to the leg portion 22. It is contemplated that the control device 76 may include any desired number of elastic members to adjust the force applied by the control device to resist or restrict movement of the foot portion 50 relative to the leg portion 22.

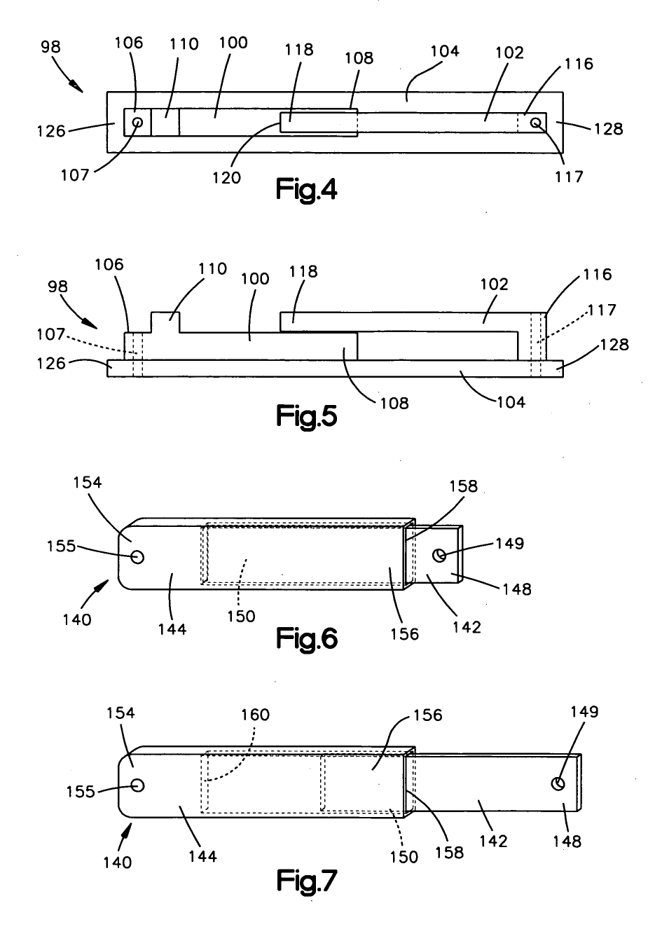

A second embodiment of a control device for controlling relative movement between the leg portion 22 and the foot portion 50 is illustrated in FIGS. 4 and 5. The control device 98 (FIGS. 4 and 5) may include first and second rigid members 100 and 102 that move relative to each other when the leg portion 22 and the foot portion 50 move relative to each other. The control device 98 may include a third elastic member 104 connected to the first and second members 100 and 102 to resist or restrict relative movement between the first and second members and between the leg and foot portions 22 and 50.

The first member 100 may have a first end 106 pivotally connected to the leg portion 22. The first end 106 may include an opening 107 through which the fastener 80 may extend. A second end 108 of the rigid member 100 may extend below the second member 102 and slidably engage the second member. The first member 100 may include an upwardly, as viewed in FIG. 5, extending stop 110 between the first and second ends 106 and 108. The stop 110 may be engageable with the second member 102 to prevent movement of the foot portion 50 relative to the leg portion 22 in the first direction, such as counterclockwise or dorsiflexion. The stop 110 may be located in any desired position along the first member 100.

The second member 102 may have a first end 116 pivotally connected to the foot portion 50. The first end 116 may include an opening 117 through which the fastener 66 may extend. A second end 118 may slidably engage the first member 100. The second end 118 may include an end surface 120 engageable with the stop 110 to stop relative movement between the first and second members.

The third member 104 may have a first end 126 pivotally connected to the leg portion 22 by the fastener 80 that connects the first end 106 of the first member 100 to the leg portion. The third member 104 may have a second end 128 pivotally connected to the foot portion 50 by the fastener 66 that connects the first end 116 of the second member 102 to the foot portion. The elastic member 104 may resist or restrict movement of the first end 116 of the second member 102 away from the first end 106 of the first member. Accordingly, the elastic member 104 may resist or restrict movement of the foot portion 50 relative to the leg portion 22 in the second direction, such as planter flexion or clockwise, as viewed in FIGS. 1 and 2.

The control device 98 may resist or restrict movement of the foot portion 50 relative to the leg portion 22 from the predetermined position in the second direction, such as plantar flexion or clockwise, as viewed in FIGS. 1 and 2 until the third member 104 is fully extended. The third member 104 may stretch and apply a force between the leg portion 22 and the foot portion 50 during movement of the foot portion in the second direction to resist or restrict movement in the second direction. The third member 104 may urge the foot portion 50 to move in the first direction when the third member stretches. The third member 104 may prevent or stop further movement of the foot portion 50 relative to the leg portion 22 in the second direction when the third member is fully extended. The control device 98 may permit free movement of the foot portion 50 relative to the leg portion 22 from the predetermined position in the first direction, such as dorsiflexion or counterclockwise, as viewed in FIGS. 1 and 2 until the end surface 120 of the second member 102 engages the stop 110 on the first member 100. Once the end surface 120 engages the stop 110, the foot portion 50 is prevented from moving further in the first direction.

It is contemplated that the end surface 120 may engage the stop 110 when the foot portion 50 is in the predetermined position relative to the leg portion 22. Accordingly, the control device 98 may prevent movement of the foot portion 50 relative to the leg portion 22 in the first direction. It is also contemplated that a member, such as a spring member, may urge the second member 102 to move away from the stop 110.

A third embodiment of an exemplary control device is illustrated in FIGS. 6 and 7. The control device 140 may include first and second rigid members 142 and 144 that move relative to each other. The first member 142 may have a first end 148 pivotally connected to the foot portion 50. The first end 148 may include an opening 149 through which the fastener 66 may extend. A second end 150 of the rigid member 142 may extend into the second member 144 in a telescoping manner.

The second member 144 may have a first end 154 pivotally connected to the leg portion 22. The first end 154 may include an opening 155 through which the fastener 80 may extend. A second end 156 may have an axially extending opening 158 into which the second end 150 of the first member 142 extends. A stop surface 160 may at least partially define the opening 158. The second end 150 of the first member 142 may engage the stop surface 160 to prevent further movement of the first member into the opening 158.

The control device 140 may permit free movement of the foot portion 50 relative to the leg portion 22 from the predetermined position in the first direction, such as dorsiflexion or counterclockwise, as viewed in FIGS. 1 and 2, until the second end 150 of the first member 144 engages the stop surface 160. The control device 140 may stop further movement of the foot portion 50 relative to the leg portion 22 in the first direction when the second end 150 of the first member 144 engages the stop surface 160. Accordingly, the stop surface 160 may be a stop to further movement of the foot portion 50 in the first direction. The control device 140 may permit free movement of the foot portion 50 relative to the leg portion 22 from the predetermined position in the second direction, such as plantar flexion or clockwise, as viewed in FIGS. 1 and 2.

It is contemplated that the second end 150 of the first member 142 may engage the stop surface 160 when the foot portion 50 is in the predetermined position relative to the leg portion 22. Accordingly, the control device 140 may prevent movement of the foot portion 50 in the first direction relative to the leg portion 22. It is also contemplated that a member, such as a spring member or elastic member, may urge the first member 142 to move toward the stop surface 160. It is contemplated that a member, such as a spring member, may urge the first member 142 to move away from the stop surface 100.

The orthosis 10 may be used to control plantar flexion and/or dorsiflexion. The orthosis 10 may resist, stop, prevent, or permit free plantar flexion. The orthosis 10 may resist, stop, prevent, or permit free dorsiflexion. Accordingly, any desired control device 76, 98 or 140 may be connected between the leg portion 22 and the foot portion 50 without altering the shape of the orthosis.

From the above description of the invention, those skilled in the art will perceive improvements, changes and modifications. Such improvements, changes and modifications within the skill of the art are intended to be covered by the appended claims.

Having described the invention, the following is claimed:

1. An orthosis connectable with a patient comprising:
a first portion connectable with a leg of the patient;
a second portion connectable with a foot of the patient, said second portion being movable relative to said first portion about a pivot axis spaced from a plantar/dorsal pivot axis of the patient; and
a control device that controls relative movement between said first and second portions about said pivot axis, said control device being connected with an anterior portion of the first portion and an upper portion of said second portion, said control device extending from said anterior portion of said first portion to said upper portion of said second portion.

2. An orthosis as set forth in claim 1 wherein said control device resists movement of said second portion in a first direction relative to said first portion.

3. An orthosis as set forth in claim 1 wherein said control device includes a stop that stops movement of said second portion in a first direction relative to said first portion.

4. An orthosis as set forth in claim 1 wherein said control device includes a flexible member.

5. An orthosis as set forth in claim 4 wherein said flexible member applies a force to said first and second portions to resist relative movement between said first and second portions.

6. An orthosis as set forth in claim 1 wherein said control device includes a first member connected with said first portion and a second member movable relative to said first member connected with said second portion, said first and second members being engageable with each other.

7. An orthosis as set forth in claim 6 wherein said first member has a stop portion that stops movement of said second portion relative to said first portion, said second member being engageable with said stop.

8. An orthosis as set forth in claim 6 wherein a third member applies a force to said first and second portions to resist relative movement of said second portion in a first direction relative to said second portion.

9. An orthosis as set forth in claim 8 wherein said third member is connected with said first and second members.

10. An orthosis as set forth in claim 6 wherein said first member includes a portion extending into an opening in said second member.

11. An orthosis as set forth in claim 10 wherein said first and second members telescope relative to each other.

12. An orthosis as set forth in claim 1 wherein a first end of said control device in pivotally connected with said first portion and a second end of said control device in pivotally connected with said second portion.

13. An orthosis as set forth in claim 1 wherein said first and second portions are formed as one-piece.

14. An orthosis as set forth in claim 1 wherein a connecting portion interconnects said first and second portions, said connecting portion being extendable about a heel of the patient and spaced from the plantar/dorsal pivot axis of the patient.

15. An orthosis as set forth in claim 14 wherein said first, second, and connecting portions are formed as one piece.

16. An orthosis as set forth in claim 14 wherein said connecting portion is resilient to permit relative movement between said first and second portions.

17. An orthosis as set forth in claim 1 wherein a fastener connects said control device with a portion of one of said first and second portions, said fastener connecting a connecting element that connects said apparatus to the patient to said portion of said one of said first and second portions.

18. An orthosis as set forth in claim 1 wherein said first end of said control device is connected to said anterior portion of said first portion at a location proximal to a plantar/dorsal pivot axis of the patient, said control device being connected to said upper portion of said second portion at a location distal to the a plantar/dorsal pivot axis.

19. An orthosis as set forth in claim 1 wherein said control device includes first and second control members connected to opposite sides of said orthosis.
